(19) United States
(12) Patent Application Publication
(10) Pub. No.: US 2015/0250543 A1
(43) Pub. Date: Sep. 10, 2015

(54) MULTI-WAVELENGTH LASER DEVICE FOR SKIN TREATMENT

(71) Applicant: Syneron Medical Ltd., Yoqneam IIIit (IL)

(72) Inventors: ROY ISRAELI, Haifa (IL); Avram HUPPERT, Kfar Yona (IL); Shmuel EISENMANN, Pardes Chana - Karkur (IL)

(21) Appl. No.: 14/620,188

(22) Filed: Feb. 12, 2015

Related U.S. Application Data
(60) Provisional application No. 61/949,274, filed on Mar. 7, 2014.

(51) Int. Cl. A61B 18/20

(52) U.S. Cl. CPC A61B 18/203; A61B 2018/207; A61B 2018/202; A61B 2018/00452

(57) ABSTRACT

Disclosed is a hand piece for skin treatment that includes at least two laser diode bars, with each bar configured to emit a laser beam with a different wavelength.

### TECHNOLOGY FIELD

The laser diodes stack relates generally to a device for skin treatment using electro-magnetic radiation. More particularly, the laser diodes stack relates to a device including a number of solid-state lasers operating at different wavelengths and produced as one monolithic device.

### BACKGROUND

Lasers are widely used in dermatological applications for the treatment of various skin anomalies, deficiencies, etc., such as hair removal, removal of pigmented lesions, removal of tattoos, treatment and removal of vascular lesions, alleviation of wrinkles, treatment of acne, and provision of skin tightening. Dermatological laser treatments are typically based on selective targeting of a chromophore in the skin by the application of laser light at an appropriate choice of wavelength and pulse duration. Because different skin deficiencies may include different chromophores, devices to provide skin treatment should include a plurality of different sources light sources that can be provisioned at a variety of wavelengths. It has been proven that lasers typically provide better treatment results than broadband light sources, however; laser devices typically provide a single wavelength of light. Because of this limitation in laser devices, most of the skin treatment products include a number of lasers. Inclusion of several different lasers into one product increases the complexity and cost of the product.

The lasers typically used for skin treatment are Alexandrite lasers, Nd:YAG lasers; Erbium lasers and different gas lasers. Each laser has its individual power supply, control and optics. The power supply is typically located in a separate housing. The optics, which may include a fiber optics light guide or an articulated arm, generally direct a laser beam or laser energy, which may includes different wavelengths of laser energy, to a hand piecehand piece. A caregiver applies to the hand piece to a target segment of the skin to be treated. A cable, which may be coupled between the hand piece and a source of power and laser energy and which may include the fiber optics light guide and/or other optics and some electrical cabling, can be cumbersome and function to reduce the ease of handling of the hand piece and caregiver freedom.

### BRIEF SUMMARY

Disclosed is a hand piece for skin treatment that includes at least two laser sources, such as diode bars, with each source configured to emit a laser beam with at least one characteristic that is unique from the other laser beams, such as having a different wavelength, intensity, duration, pulsing configuration, etc. The hand piece also includes a wavelength-combining device, the wavelength-combining device configured to receive laser signals having different characteristics, such as different wavelengths, combine the laser signals into a homogienized laser beam containing the a combination of the differing characteristics, such as the different wavelength. A controller device could be included in the hand piece or a machine to which the hand piece is connected or otherwise coupled. The controller device is configured to control the laser diode bars and in particular, the power that each of the laser diode bars emit. Each of the laser diode bars may be individually addressed and controlled. The amount of power for each of the laser beams, such as laser beam at a particular wavelength, will be adapted according to the treated skin type (i.e. amount of melanin in the epidermis) and the target tissue type, location and absorption spectrum in order to get the safest and the highest efficacy in treating that targeted skin or tissue.

Thus, the operation of some embodiments may result in the production of a homogenized laser beam that includes at least two wavelengths selected from a plurality of wavelengths in the range of 532-1250 nm and 1400-1600 nm.

For skin treatment, embodiments of the hand piece may be applied to a segment of skin to be treated and operated to irradiate the treated skin segment by a homogenized laser beam containing different wavelengths. Following application of the homogenized laser beam to the treated skin segment the hand piece could be repositioned to treat a next skin segment.

### BRIEF DESCRIPTION OF THE DRAWINGS

The features and advantages of various embodiments of the hand piece will become apparent from the following detailed description, which is to be read in conjunction with the accompanying drawings, in which:

FIG. 1 is a simplified example of an existing laser diode array or bar;

FIG. 2 is a simplified example of an existing laser diodes stack;

FIG. 3A is a simplified illustration of a laser diode bar assembly according to an example;

FIG. 3B is a more complex illustration of the laser diode bar assembly of FIG. 3A;

FIGS. 4A and 4B are examples of laser beam combiners configured to combine and homogenize different wavelengths emitted by laser diode bars;

FIG. 5 is a schematic illustration of an example of a laser diodes stack including four laser diode bars with each laser diode bar emitting a different wavelength; and

FIG. 6 is a schematic illustration of an example of a hand piece of a skin treatment apparatus employing the present laser diodes stack.

### DETAILED DESCRIPTION

Semiconductor lasers are available in a variety of wavelengths and could be used as substitutes for Alexandrite, Nd:YAG, Erbium, and other similar solid state or gas lasers. However, each semiconductor laser also emits in a relatively narrow wavelength range. Thus, if it is desired to have a laser beam that covers a wider wavelength range, a dedicated optical system is required for combining laser beams of different wavelengths into one laser beam. For instance, such a combined signal could be used for skin treatment. The present disclosure presents various embodiments of a device that includes a stack of semiconductor laser arrays, such as laser diode arrays or bars, and the device operates to generate and combine two or more laser beams, wherein each such laser beam differs from the other laser beams in one or more characteristics (such as wavelength) into a single laser beam suitable for skin treatment.

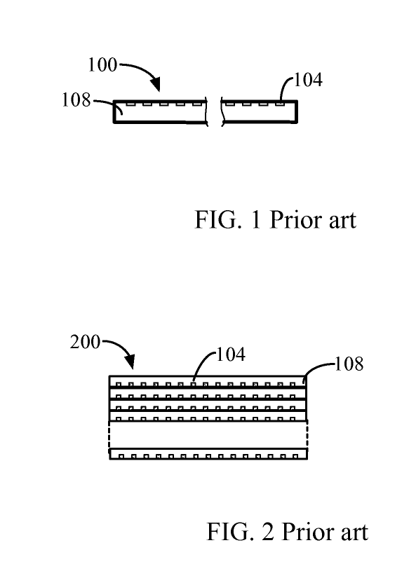

FIG. 1 is a simplified example of an existing laser diode array or bar that may be found in the prior art. A laser diode bar 100 is typically a one-dimensional array of a plurality of individual semiconductor laser emitters or laser diodes 104 assembled on a common substrate 108. A laser diode array or bar could include a wide number of individual laser diodes 104 (such as 20, 50, 70 or more as non-limiting examples) emitting laser beams at the same wavelength. As shown in FIG. 2, a number of laser diode bars could be stacked to form what is termed as a laser diode bar stack 200. Different optical arrangements could be used to couple the laser beams emitted by each of the individual laser diodes into one high power laser beam. Existing laser diode bar stacks do not support inclusion of laser diode bars that emit laser beams at different wavelengths into one laser diode bar stack. The current disclosure provides a method and device for supporting the manufacture and coupling of laser diode stacks configured to emit laser beams at different wavelengths and combine such laser beams into one common laser beam. Advantageously, the such laser diode stacks emitting different wavelengths into one common laser beam may support miniaturization of skin treatment equipment and simplify the use of such equipment.

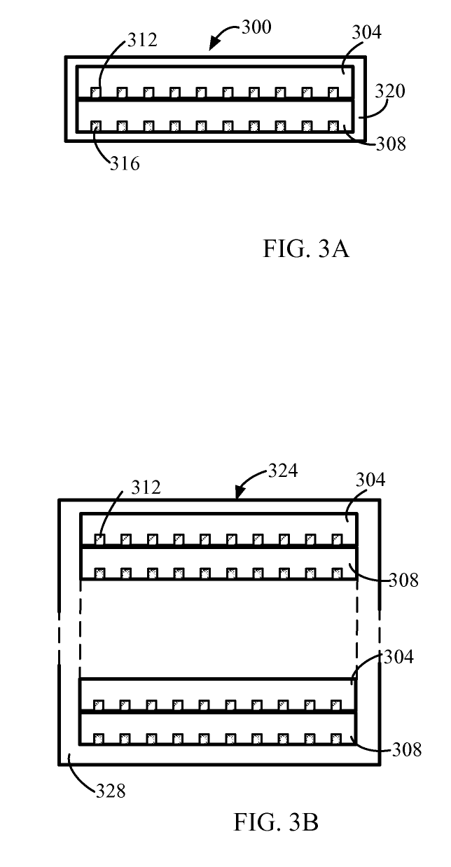

In one exemplary embodiment, as illustrated in FIG. 3A, a laser diode bar assembly or stack 300 is illustrated as including two laser diode bars 304 and 308. Each of two laser diode bars 304 and 308 includes laser emitters 312 and 316, which emit laser radiation or beams at a different wavelengths. For example, laser diode bar 304 could be configured to emit a laser radiation with a first wavelength and laser diode bar 308 could be configured to emit a laser radiation with a second wavelength. As a non-limiting example, the wavelength could be 760 nm, 775 nm, 810 nm, 905 nm, 1064 nm, 1210 nm, 1470 nm, 1540 nm and other wavelengths. Laser diode bars 304 and 308 could be mounted on a common substrate or mount 320 that could be made of a suitable heat conducting material, such as aluminum or copper, and serve as heat sink for the laser diode bar stack or assembly. In some examples, such as is illustrated in FIG. 3B, the laser diode bar assembly 324 could include more than two laser diode bars. For example, there could be four or ten, or more laser diode bars 304 emitting at a first wavelength and four or ten, or more laser diode bars 308 emitting at a second wavelength (as non-limiting examples). Each laser diodes bar could emit a laser beam having a laser power between 10 W to 120 W as a non-limiting example. The plurality of laser diode bars could be mounted on a common substrate 328, which would serve as a mount and as a heat sink.

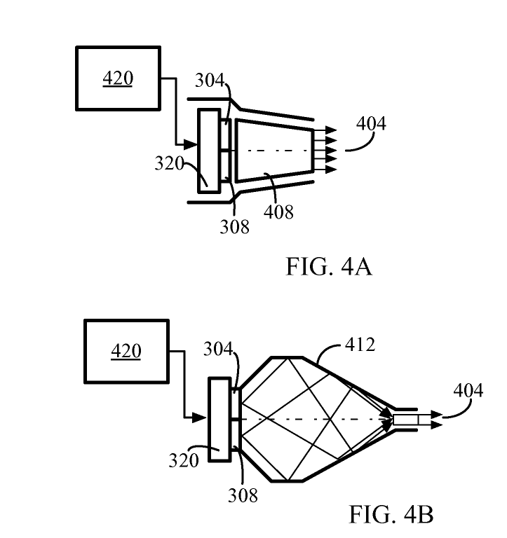

FIGS. 4A and 4B are examples of laser beam combiners configured to combine and homogenize different wavelengths emitted by laser diode bars. As shown in FIG. 4A, laser diode bar assembly or stack 300 (or 324) could also include a coupling optics that could be constructed of refractive and/or reflective optics. For example, an optical beam combiner could be configured to combine and homogenize the laser beam of different wavelengths emitted by laser diode bars 304 and 308 into a common laser beam 404. In one example, the laser beam combiner could be a refractive light guide 408 that could be a tapered trapezoidal prism or other suitable cross section prisms or mirror systems. Light guide 408 could be made from sapphire or other suitable material and have dimensions of 25 to 100 mm in length and width matching the size of the laser diode bars stack or assembly as a non-limiting example. The most widely spread width of laser diode bars is about 10 mm.

FIG. 4B illustrates a beam combiner 412 of reflective type. A combination or refractive optical elements such as prisms and reflective optical elements is also possible to use to combine laser radiation emitted by different laser diode bars.

Optical fiber could also be used to combine and homogenize the two different wavelengths emitted by laser diode bars 304 and 308 into a common laser beam 404. Each laser diode bar could be coupled into an optical fiber and the individual optical fibers could be spliced into another optical fiber that could serve as a wavelength combining device and homogenize the wavelengths delivered by the different optical fibers included in the splice.

As further illustrated in FIG. 4A and FIG. 4B, an exemplary laser diode bar stack or assembly could also include a controller device 420 configured to control the various laser diode bars, such as a first and the second laser diode bar, and the whole stack. The controller 420 could be configured to address and control each of the laser diode bars 304 or 308 individually or in unison, as well as to control even the individual laser emitters within each of the laser diode bars. The amount of power for each of the laser beams emitted by the stack or even the individual diode bar of each stack, will be adapted and controlled according to the treated skin type (i.e. amount of melanin in the epidermis) and the target tissue type, location and absorption spectrum, etc., in order to get the safest and the highest efficacy in treating that targeted skin or tissue. The amount of power for example could be 40% for the laser beam having a first wavelength and 60% for the laser beam having the second wavelength as a non-limiting example. As another non-limiting example, the power could be 10% of the second wavelength and 90% of the first wavelength. The mix of wavelengths could be adapted for treatment of different hues of fair or dark skin as determined by the amount of melanin in the epidermis. The adaptation of the power for each of the laser beams at particular wavelengths emitted by the laser bars or lasers could be manual or automatic in response for the melanin values entered by the operator, visual inspection by electronic devices such as cameras, light detectors combined with a light source to detect the amount of light reflected from the surface and thus the color of the skin, pre-entered profile information, etc.

In one example, the stack 300 of laser diode bars could be replaced by two or more fiber lasers or solid state lasers, which are coupled to a fiber or free space optics and are used to deliver to the hand piece a mix of two or more laser beams with different wavelengths at a power level of 10 W to 10 kW. Such lasers could, for example, be Alexandrite or Nd:YAG laser, Erbium lasers and different gas lasers. A frequency doubling device, such as a KTP plate (KTP is abbreviation for Potassium Titanyl Phosphate Single Crystal, (KTiOPO4)) or similar could be used to provide additional wavelengths. In some examples, angular orientation of the frequency doubling device could be used to vary and control the mix of power provided by the lasers.

In some examples, control of the amount of power in each of the wavelengths, regardless of the laser source type, is attained by adding interference filters (bandpass or high/low pass filters) designed for the wavelengths in use and operating to misalign the laser beam in order to achieve the needed energy in each of the particular wavelengths.

In some examples, the sources are at least two fiber lasers, each emitting at a different wavelength. Each of fiber laser sources could be spliced into another optical fiber that could serve as a wavelength-combining device and homogenize the wavelengths delivered by the different fiber lasers carried by different optical fibers and spliced into a single fiber.

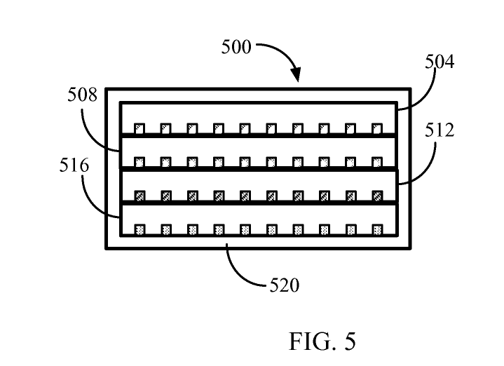

FIG. 5 is a schematic illustration of an example of a laser diodes stack including four laser diode bars with each laser diode bar emitting a different wavelength. In some examples, as shown in FIG. 5, laser diode bar stack or assembly 500 could include more than two laser diode bars, or types/configurations of laser diode bars. For example, there could be four laser diode bars 504, 508, 512, and 516 with each of the laser diode bars emitting laser beams at a different wavelength. The plurality of laser diode bars 504, 508, 512, and 516 could be mounted on a common substrate 520 and have a common beam combiner and/or controller.

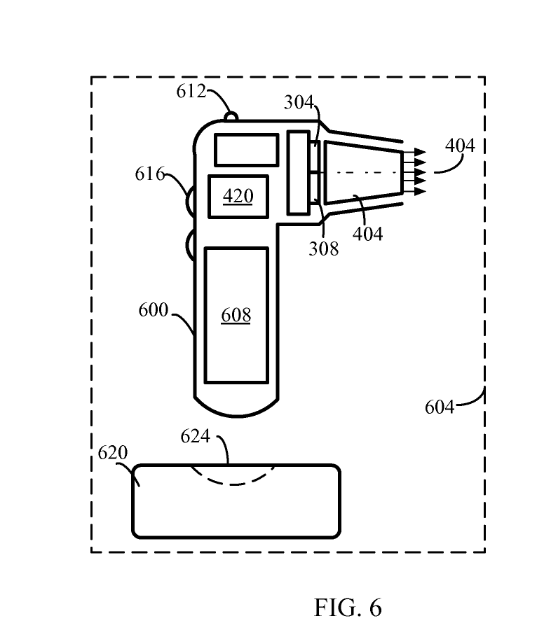

FIG. 6 is a schematic illustration of an example of a hand piece of a skin treatment apparatus employing the present laser diodes stack. Because of the relatively small size of the laser diodes stack, the stack could be mounted within a hand piece 600 of a skin treatment apparatus 604. A rechargeable battery 608 could be included in hand piece 600 and configured to provide power to the laser diode bars, controller 420 and different hand piece 600 status indicators, such as one or more LEDs 612 or display. LEDs 612 could light in a number of different colors with each color indicating the current hand piece 600 status or, a digital display may actually provide codes, numbers or words to indicate the status and/or settings. One or more button switches 616 could be configured to switch ON or OFF different functions of the hand piece 600. For charging, hand piece 600 could be inserted into a docking and charging station 620 receptacle 624. For skin treatment hand piece 600 hand piece including at least two laser diode bars, with each bar configured to emit a laser radiation with a different wavelength and a wavelength combining device, could be applied to a segment of skin to be treated and operated to irradiate the segment of skin to be treated by an homogenized laser beam containing a combination of laser beams with different wavelengths and following application of homogenized laser beam to the treated skin segment repositioning the hand piece to treat a next skin segment. The amount of energy coupled into each wavelength could be set and controlled according to the skin type and target chromophores to be treated. Operating the hand piece to irradiate the treated skin segment by an homogenized laser beam containing the different wavelengths and following application of homogenized laser beam of proper power to the treated skin segment, repositioning the hand piece to treat a next skin segment.

Laser diode bar stacks could be implemented as an exchangeable stack combining different wavelength combinations and could be inserted according to a desired skin treatment protocol. Two wavelengths simultaneously irradiating the skin segment to be treated could be selected for example to provide hair removal and wrinkle treatment or acne treatment and hair removal. For example, a combination of laser diode bars emitting at 760 nm and 1064 or 808 nm and 1064 nm could be used for hair removal. Other combinations most appropriate for a different desired treatment could be used. In addition, a single laser diode bar stack may include multiple types of laser diode bars, each transmitting laser beams at a different wavelength. By controlling the power delivered from each laser diode bar in the stack, different combinations of laser beam wavelengths can be generated from a single laser diode bar stack, thus allowing the hand piece to be adjusted, either manually or automatically for different skin types, treatment types, etc.

It should be recognized that a number of variations of the above-described examples will be obvious to one of ordinary skill. Accordingly, the apparatus and method are not to be limited by those specific examples and methods as shown and described herein. Rather, the scope of the apparatus and method is to be defined by the following claims and their equivalents.

What is claimed:

1. An assembly comprising:
at least one laser diode bar configured to emit at least a first laser beam having a first wavelength;
at least one laser diode bar configured to emit at least a second laser beam having a second wavelength;
a wavelength combining device, the wavelength combining device is configured to receive the at least first laser beam and the at least second laser beam at the first and second wavelength respectively, and create a combined homogenized laser beam containing the first and the second wavelength.

2. The assembly according to claim 1, wherein the laser diode bars are mounted on a common substrate and wherein the substrate serves as a heat sink.

3. The assembly of claim 1, further comprising a controller device configured to control the laser diode bars and wherein each of the laser diode bars is configured to be individually addressed and controlled.

4. The assembly of claim 1, wherein each of the laser diode bars are semiconductor laser bars and at least two semiconductor laser bars emit at least at one of the wavelengths of 775 nm, 810 nm, 905 nm, 1064 nm, 1400 nm and 1600 nm.

5. The assembly of claim 1, wherein the wavelength combining device is one of a group consisting of a light guide, a prism beam combiner, and a reflective beam combiner.

6. The assembly of claim 1, wherein the wavelength combining device is at least one of a group consisting of a reflective optics and refractive optics.

7. The assembly of claim 1, wherein the wavelength combining device is at least one optical fiber.

8. A hand piece for skin treatment comprising
at least two laser diode bars, with each bar configured to emit one or more laser beams and, the one or more laser beams for each laser diode bar has a different wavelength from the one or more laser bears of the other laser diode bars;
a wavelength combining device, the wavelength combining device configured to receive laser beams of at least a first and a second wavelength and combine them into a homogenized laser radiation beam containing at least the first and second wavelength.

9. The hand piece of claim 8, further comprising a controller device configured to control at least a first and a second laser diode bars and wherein each of the laser diode bars is individually addressed and controlled.

10. The hand piece of claim 8, wherein the homogenized laser radiation beam includes at least two wavelengths selected from a group of wavelengths consisting of 775 nm, 810 nm, 905 nm, 1064 nm, 1400 nm and 1600 nm.

11. The hand piece of claim 8, wherein the wavelength combining device is one of a group consisting of a light guide, a prism beam combiner, and a reflective beam combiner.

12. The hand piece of claim 8, wherein the wavelength combining device is at least one optical fiber.

13. A method of skin treatment comprising:
applying to a segment of skin to be treated a hand piece, wherein the hand piece includes:
at least two laser diode bars, with each bar configured to emit a laser radiation with a different wavelength; and
a wavelength combining device, the wavelength combining device configured to receive laser radiation with different wavelengths and combine them into a homogenized laser beam containing different wavelengths;
operating the hand piece to irradiate the segment of skin by a homogenized laser beam containing the different wavelengths; and
following application of homogenized laser beam to the segment of skin, reposition the hand piece to treat a next segment of skin.

14. A method of skin treatment comprising:
applying to a segment of skin to be treated a hand piece, the hand piece including:
at least two laser diode bars, with each bar configured to emit a laser radiation with a different wavelength; and
a wavelength combining device, the wavelength combining device configured to receive laser radiation with different wavelengths and combine them into a homogenized laser beam containing different wavelengths;
controlling the amount of energy in each wavelength according to the skin type and target chromophores of the segment of skin to be treated; and
operating the hand piece to irradiate the segment of skin by a homogenized laser beam containing the different wavelengths; and
following application of homogenized laser beam to the treated skin segment, repositioning the hand piece to treat a next skin segment.

15. The method of skin treatment of claim 14, wherein the wavelength combining device receives laser radiation with different wavelengths and combines the different wavelengths into a homogenized laser beam containing different wavelengths.

16. The method of skin treatment of claim 14, wherein the hand piece further comprises a controller and wherein the controller is configured to control the amount of energy in each wavelength according to the skin type and target chromophores of the segment of skin.

17. The method of claim 14, wherein the amount of power for each of the wavelengths is adapted according to the skin type of the segment of skin.
